(19) United States
(12) Patent Application Publication
(10) Pub. No.: US 2018/0085540 A1
(43) Pub. Date: Mar. 29, 2018

(54) INHALER WITH ONE OR MORE VISUAL SENSORS

(71) Applicant: QUALCOMM Incorporated, San Diego, CA (US)

(72) Inventors: Eugene Dantsker, San Diego, CA (US); Evgeni Gousev, Saratoga, CA (US)

(21) Appl. No.: 15/669,066

(22) Filed: Aug. 4, 2017

Related U.S. Application Data
(60) Provisional application No. 62/401,097, filed on Sep. 28, 2016.

(51) Int. Cl. A61M 16/00; A61M 15/00; A61B 5/00; G06F 19/00

(52) U.S. Cl. CPC A61M 16/0003; A61M 15/009; A61M 15/0081; A61M 2230/40; G06F 19/3462; A61M 2205/52; A61B 5/4833

(57) ABSTRACT

Techniques disclosed herein provide for increased accuracy of information regarding the administration of medicine via an inhaler by obtaining data from a variety of data sources, including a visual sensor of the inhaler. Data can then be fused to make a determination of the effectiveness of how the medicine was administered, and an indication of the determined effectiveness can be relayed to another device.

### RELATED APPLICATIONS

This application claims the benefit of U.S. Provisional Application No. 62/401,097, filed Sep. 28, 2016, entitled “MEDICAL DISPENSER WITH ONE OR MORE VISUAL SENSORS”, of which is assigned to the assignee hereof, and incorporated herein in its entirety by reference.

### BACKGROUND

The drug delivery actuated by the patient via an inhaler may be tracked to help ensure medication compliance, adherence, and persistence by health care providers and/or other stakeholders. In recent years, inhalers have increasingly been equipped with sensors and/or other components to help with this tracking. Even so, these inhalers generally lack the ability to confirm that the drug, in fact, was properly taken by the patient.

### SUMMARY

Techniques disclosed herein provide for increased accuracy of information regarding the administration of medicine via an inhaler by obtaining data from a variety of data sources, including a visual sensor of the inhaler. Data can then be fused to make a determination of the effectiveness of how the medicine was administered, and an indication of the determined effectiveness can be relayed to another device.

An example method of operating an inhaler with a visual sensor, according to the description, comprises obtaining, at the inhaler, information from the visual sensor indicative of the presence of one or more facial features, obtaining orientation information indicative of an orientation of the inhaler, receiving an indication that a drug has been administered by the inhaler, and making a determination of an effectiveness of how the drug was administered with the inhaler based, at least in part, on the information from the visual sensor, the orientation information, and the indication that the drug has been administered. The method further comprises sending an indication of the determined effectiveness from the inhaler to another device.

The example method may comprise one or more of the following features. The information regarding the presence of one or more facial features further can comprise information regarding orientation, size, proximity, or location of the one or more facial features, or any combination thereof. The method may further comprise sending, from the inhaler to the other device, an indication of a time at which the drug was administered. The orientation information may be obtained from an accelerometer, a gyroscope, and/or the visual sensor. Making the determination of the effectiveness of how the drug was administered with the inhaler may be further based on information from a microphone. The information from the microphone may be indicative of timing of inhalation, duration of inhalation, or both. Making the determination of the effectiveness of how the drug was administered with the inhaler may be further based on information received from a light sensor. Making the determination of the effectiveness of how the drug was administered with the inhaler may be further based on information received from a sensor configured to measure resistance, conductance, capacitance, or any combination thereof. The method may further comprise, in response to obtaining the information from a visual sensor regarding the presence of one or more facial features, releasing a locking mechanism of the inhaler to enable administration of the drug. The information indicative of the presence of the one or more facial features may comprise information indicative of a facial recognition event. Making the determination of the effectiveness of how the drug was administered with the inhaler may be performed by a processor that awakens from a low-power mode based, at least in part, on the obtaining the information from the visual sensor indicative of the presence of the one or more facial features. The indication of the determined effectiveness may be based on a numerical scale.

An example inhaler, according to the description, comprises a visual sensor configured to visually detect the presence of one or more facial features of a person using the inhaler, an orientation sensor configured to determine an orientation of the inhaler, a wireless communication interface, and a unit communicatively coupled with the visual sensor, the orientation sensor, and the wireless communication interface. The processing unit is configured to obtain, from the visual sensor, an indication of the presence of the one or more facial features, obtain, from the orientation sensor, orientation information indicative of the orientation of the inhaler, receive an indication that a drug has been administered by the inhaler, and make a determination of an effectiveness of how the drug was administered with the inhaler based, at least in part, on the information from the visual sensor, the orientation information, and the indication that the drug has been administered. The processing unit is further configured to send an indication of the determined effectiveness, via the wireless communication interface, from the inhaler to another device.

The inhaler may further comprise one or more the following features. The visual sensor may be configured to provide information regarding the presence of one or more facial features that comprises information regarding orientation, size, proximity, or location of the one or more facial features, or any combination thereof. The processing unit may be further configured to send, from the inhaler to the other device, an indication of a time at which the drug was administered. The orientation sensor may comprise an accelerometer, a gyroscope, and/or the visual sensor. The inhaler may further comprise a microphone, and the processing unit may be further configured to make the determination of the effectiveness of how the drug was administered with the inhaler further based on information from the microphone. The information from the microphone may be indicative of timing of inhalation, duration of inhalation, or both. The inhaler may further comprise comprising a light sensor, and the processing unit may be further configured to make the determination of the effectiveness of how the drug was administered with the inhaler further based on information received from the light sensor. The inhaler may further comprise a skin-detection sensor configured to measure resistance, conductance, capacitance, or any combination thereof, and the processing unit may be further configured to make the determination of the effectiveness of how the drug was administered with the inhaler further based on information received from a sensor. The inhaler may further comprise a locking mechanism, and in the processing unit may be further configured to, in response to obtaining the information from a visual sensor regarding the presence of one or more facial features, release the locking mechanism to enable administration of the drug. The processing unit may be configured to awaken from a low-power mode based, at least in part, on the obtaining the information from the visual sensor indicative of the presence of the one or more facial features. The processing unit may be configured to determine the indication of the determined effectiveness based on a numerical scale.

An example drug-administering apparatus, according to the description, comprises means for obtaining information from visual-sensing means indicative of the presence of one or more facial features, means for obtaining orientation information indicative of an orientation of the drug-administering apparatus, means for receiving an indication that a drug has been administered by the drug-administering apparatus, and means for making a determination of an effectiveness of how the drug was administered with the drug-administering apparatus based, at least in part, on the information from the visual-sensing means, the orientation information, and the indication that the drug has been administered. The drug-administering apparatus further comprises means for wirelessly sending an indication of the determined effectiveness from the drug-administering apparatus to a separate device.

The drug-administering apparatus may comprise one or more the following features. The drug-administering apparatus may comprise means for sending, from the drug-administering apparatus to the separate device, an indication of a time at which the drug was administered. The means for making the determination of the effectiveness of how the drug was administered with the administering apparatus may comprise means for further basing the determination on information received from means for detecting sound. The means for making the determination of the effectiveness of how the drug was administered with the administering apparatus further may comprise means for further basing the determination on information received from means for detecting light. The means for making the determination of the effectiveness of how the drug was administered with the administering apparatus further may comprise means for further basing the determination on information received from means for detecting skin. The drug-administering apparatus further may comprise, means for, in response to obtaining the information from a visual sensor regarding the presence of one or more facial features, releasing a locking mechanism of the drug-administering apparatus to enable administration of the drug.

An example non-transitory computer-readable medium, according to the description, may have instructions embedded thereon for operating an inhaler with a visual sensor, where the instructions comprising computer code for obtaining information from the visual sensor indicative of the presence of one or more facial features, obtaining orientation information indicative of an orientation of the inhaler, receiving an indication that a drug has been administered by the inhaler, making a determination of an effectiveness of how the drug was administered with the inhaler based, at least in part, on the information from the visual sensor, the orientation information, and the indication that the drug has been administered. The instructions may further have computer code for sending an indication of the determined effectiveness from the inhaler to another device.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is an example system for providing information about the administration of medicine by an inhaler to one or more stakeholders.

FIG. 2 is a simplified perspective view of an inhaler, according to embodiments herein.

FIGS. 3A-3B are simplified block diagrams of electrical components of an inhaler, according to embodiments.

FIG. 4 is a flow diagram illustrating an embodiment of a method of determining proper administration of a drug by an inhaler, according to one embodiment.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

Patients suffering from respiratory and pulmonary ailments often take medications administered via inhalation. The drug delivery mechanism is contained in an inhaler that is inserted into the mouth or nose and actuated by the patient thereby releasing the inhalant medications into the respiratory system.

In recent years, inhalers have become modified with electronic modules in order to track and report medication compliance, adherence, and persistence. These “smart” inhalers keep track of such parameters as the date & time of the inhalation, the orientation of the inhaler and the volume of the released drug. While these inhalers are leading to better tracking of drug release, they still lack the ability to confirm that the drug, in fact, was ingested by the patient. In an obvious example, an inhaler can be deployed with the drug released into the air rather than into the mouth or nose of the patient. In this case, a “smart” inhaler would still register “use” even though the drug was not consumed.

Establishing that the right dose of the right drug is administered to the right patient at the right time via the right route can be important not only to the person taking the drug, but to many other entities as well. Other stakeholders that have an interest in this information include, for example, the doctor that prescribed and/or is overseeing the administration of the drug, a manufacturer of the drug, an insurance provider (and/or other payer), a government health agency and/or other health organization, and/or the like. For each of the stakeholders, the use and/or misuse of a drug may impact the decisions of a stakeholder with regard to the drug. For example, a drug that is consistently misused may impact whether or how an insurance provider is willing to pay for the use of the drug, and/or how to adjust premiums for patients that may consistently misuse drugs. It may also prompt a manufacturer to modify the design of the inhaler or determine another means of administration of the drug to help reduce the misuse of its administration. All stakeholders may be impacted by use/misuse information in determining how effective a drug for a population of patients.

Embodiments disclosed herein help increase the accuracy of this information provided to the stakeholders by utilizing a low cost, low power visual sensor on the inhaler capable of detecting that the face and/or various facial features of the patient are in the correct proximity to the inhaler when it is deployed. In short, the visual sensor can discern whether the inhaler is used in such a manner that the drug is being consumed by a patient or merely deployed into the air (whether by accident or intentionally). In some embodiments, the visual sensor may be able to detect certain facial features of the patient, such as irises, in order to confirm the patient's identity. In some embodiments, the visual sensor can also provide for a feature that can act as a “locksafe” mechanism that prevents the drug from being deployed if the inhaler does not detect user features in the right position or does not detect an authorized user based on user features using user recognition algorithms (e.g., feature recognition to detect and/or identify a user). For example, user authorization may be used to disengage the locksafe mechanism where authorization may include proper user orientation and/or user orientation relative to the medical dispenser.

It can be noted that, as used herein, the term “facial recognition” can refer to any form of user identification using facial features. Accordingly, such facial recognition may comprise not only determining the identity of a user based on recognized facial features, but also matching iris and/or other features on a face to corresponding features (e.g., stored in a database) of one or more authorized users.

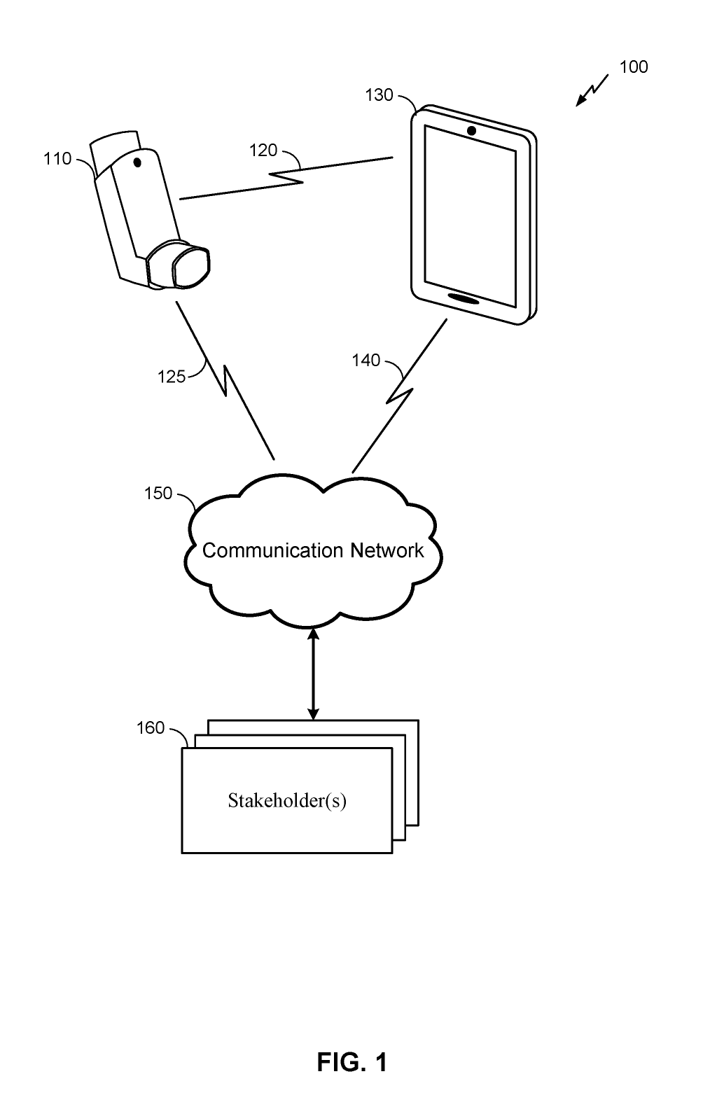

FIG. 1 is an example system 100 for providing information about the administration of medicine by an inhaler 110 to one or more stakeholders 160. Here, the system 100 may comprise an inhaler 110 as described herein, along with a connecting device 130, communication network 150, and the stakeholder(s) 160. It will be understood, however, that embodiments of a system 100 may include a different configuration of components, the addition and/or omission of various components, and/or the like, depending on desired functionality.

The inhaler 110, which is described in more detail herein below, is used to administer a drug to a patient. The patient usually administers the drug to him/herself, typically by engaging a physical mechanism (e.g., pressing down on a canister contained by the inhaler 110 or actuating a release mechanism) while inhaling. Once the drug is administered, the inhaler 110 can then register, store and transmit data associated with the administration of the drug to the connecting device 130. This data can be transmitted wirelessly via a wireless communication link 120, using any of a variety of wireless technologies as described in further detail below.

It can be noted that, although embodiments described herein are directed toward an inhaler, other embodiments are not so limited. The techniques disclosed herein may generally apply to a mobile medical dispenser that includes one or more visual sensors as described herein. Medicine may be dispensed using means other than inhalation (e.g., medicine that is taken orally, transdermally, intravenously, etc.), depending on desired functionality. Examples of a mobile medical dispenser that includes one or more visual sensors can include, without limitation, pill/capsule containers, liquid medicine containers, syringes, and the like.

It can be further noted that although embodiments described herein are directed toward detecting facial features, other embodiments are not so limited. Embodiments can include detecting features of and/or related to a user (in addition to or instead of facial features) that can be used to detect and/or identify the user prior to the use. For example, a visual sensor may be used to capture an image of the user's fingerprint, which may be used to determine authorization.

The connecting device 130 may comprise any of a variety of electronic devices capable of receiving information from the inhaler 110 and communicating information to the stakeholder(s) 160 via the communication network 150. This can include, for example, a mobile phone, tablet, laptop, portable media player, personal computer, or similar device. In some embodiments, the connecting device 130 may comprise a specialized device utilized for purposes of conveying information from the inhaler (and possibly other medical devices) to the stakeholder(s) 160.

The connecting device 130 may execute an application to provide the data processing and/or relaying functionality illustrated in FIG. 1. In some embodiments, the application may be configurable by a user, or may simply be downloaded to the connecting device 130 and executed automatically. The application may help establish the communication link 120 between the inhaler 110 and the connecting device 130, which may or may not require input from the user, depending on desired functionality. In some embodiments, the application may provide instructions to a user on proper use of the inhaler 110 and/or feedback to a user when improper use of the inhaler 110 is detected. As indicated below, in embodiments where facial recognition is utilized, the connecting device 130 may further be used to capture an image of the user (either with a camera of the connecting device 130 or using a visual sensor of the inhaler 110) for facial recognition. Additional and/or alternative functionality of an application executed by the connecting device 130 may be utilized as desired. (As noted elsewhere herein, such functionality includes simple relaying of the data to a remote destination or interacting with the patient about the drug administration such as confirmation and user feedback.)

The communication network 150 may comprise any of a variety of data communication networks, depending on desired functionality. The communication network 150 can include any combination of radio frequency (RF), optical fiber, satellite, and/or other wireless and/or wired communication technologies. In some embodiments, the communication network 150 can comprise the Internet and/or different data networks may comprise various network types, including cellular networks, Wi-Fi® networks, etc. These types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (CatM) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

The communication link 140 between the connecting device 130 and the communication network 150 can vary, depending on the technologies utilized by these components of the system 100. For embodiments where the connecting device 130 is a mobile phone, for example, the communication link 140 may comprise a wireless communication link utilizing the mobile phone's cellular or Wi-Fi® functionality. In embodiments where the connecting device 130 is a personal computer, the communication link 140 may comprise a wired communication link that accesses the medication network 150 via a cable or digital subscriber line (DSL) modem.

It can be noted that some embodiments may not utilize a connecting device 130 to relay data to the communication network 150. In such embodiments, the inhaler 110 (or other mobile medical dispenser) may connect directly to the communication network 150 (as shown in FIG. 1 by communication link 125, which may be used in addition to or as an alternative to communication link 120). For example, the inhaler 110 may comprise a Long Term Evolution (LTE) category M (CatM) device, NarrowBand IoT (NB-IoT), or other Low Power Wide Area Network (LPWAN). Additionally or alternatively, the inhaler 150 may comprise wireless similar to the corresponding functionality of the connecting device 130 described above. In such embodiments, the communication network may additionally or alternatively comprise a Bluetooth Mesh network (such as CSRMesh), a WiFi network, Zigbee, or WWAN (such as LTE, including CATM, or 5G).

As noted above, the stakeholder(s) 160 may include any of a variety of entities with an interest in the proper administration of medicine by the inhaler 110. This can include an individual practitioner (e.g., a doctor or nurse), a hospital, a drug manufacturer, an insurance provider (or other payer), a government agency or other health organization, and/or the like. In some embodiments, the user of the inhaler 110 (e.g., the patient) may also be a stakeholder 160 to which information regarding the use of the inhaler is provided. Governmental health regulations and/or legal agreements between the patient and/or the stakeholder(s) 160 may apply to the dissemination of information regarding the administration of medicine by the inhaler 110 to the stakeholder(s) 160. Here, the inhaler 110 can utilize a visual sensor to help ensure the accuracy of the information disseminated to the stakeholder(s) 160

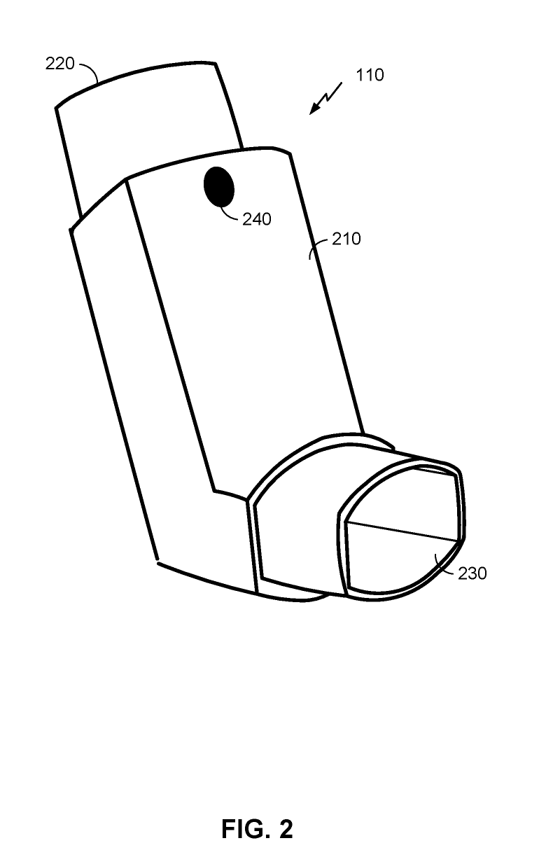

FIG. 2 is a simplified perspective view of an inhaler 110, according to embodiments herein. It can be noted, however, that inhalers can generally come in many shapes, sizes, and forms, and may enable administration of a drug through inhalation by nose and/or mouth. An inhaler 110 is generally intended to be used until the medicine contained therein runs out or when a doctor or other health care provider recommends its disuse, at which time the inhaler 110 is disposed. That said, the techniques described herein may be utilized with non-disposable inhalers.

According to embodiments herein, an inhaler 110 can comprise a canister 220 that contains the drug to be administered. Although the embodiment illustrated in FIG. 2 an exposed canister 220, alternative embodiments may have an inhaler 110 that houses medicine, with or without a canister, in a different manner. In general use, a user (e.g., a patient) will use the inhaler 110 by putting his or her mouth around the orifice 230, causing the inhaler 110 to administer the drug (e.g., by pushing downward the canister 220), and breathing in while the drug is administered via the orifice 230. Besides pressing on a canister, drug admiration can also be initiated by other mechanisms, buttons, actuators or by the inhalation process itself (e.g. by sucking on the orifice.)

According to embodiments herein, the inhaler 110 can also include a variety of additional components, which may be housed in and/or disposed on the inhaler 110, depending on desired functionality. A visual sensor 240 can be disposed on the inhaler 110 at a location where a user's features may be detected by the visual sensor 240 and can help determine whether the drug was administered properly. Here, the visual sensor 240 is located on a surface of the inhaler 100 that faces a user during the administration of the drug when the inhaler 110 is used properly. More specifically, a user's face may be within the field of view (FOV) of the visual sensor 240 immediately before, during, and/or immediately after proper administration of the drug. Accordingly, the visual sensor 240 can enable the inhaler 110 to perform facial detection and/or facial recognition of the user to help determine whether the inhaler 110 was used properly. It can be further noted that the inhaler may include a plurality of visual sensors that may be disposed elsewhere on the inhaler 110 to help determine additional or alternative visual features in the environment of the inhaler 110.

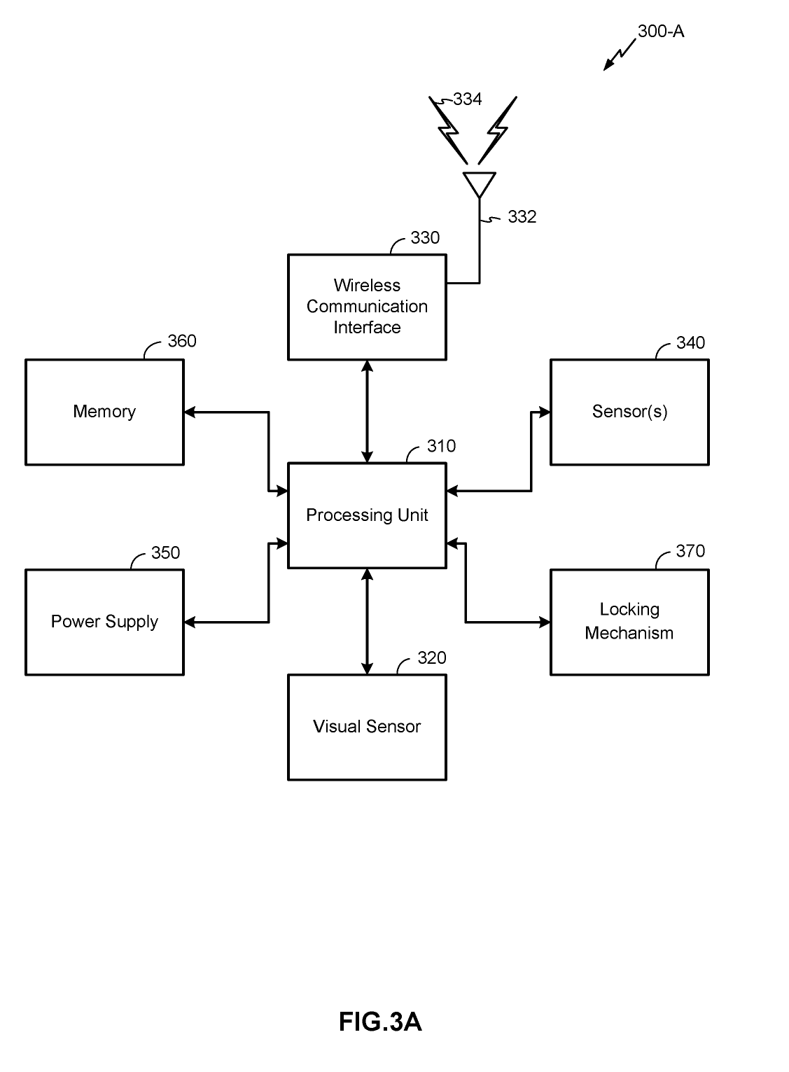

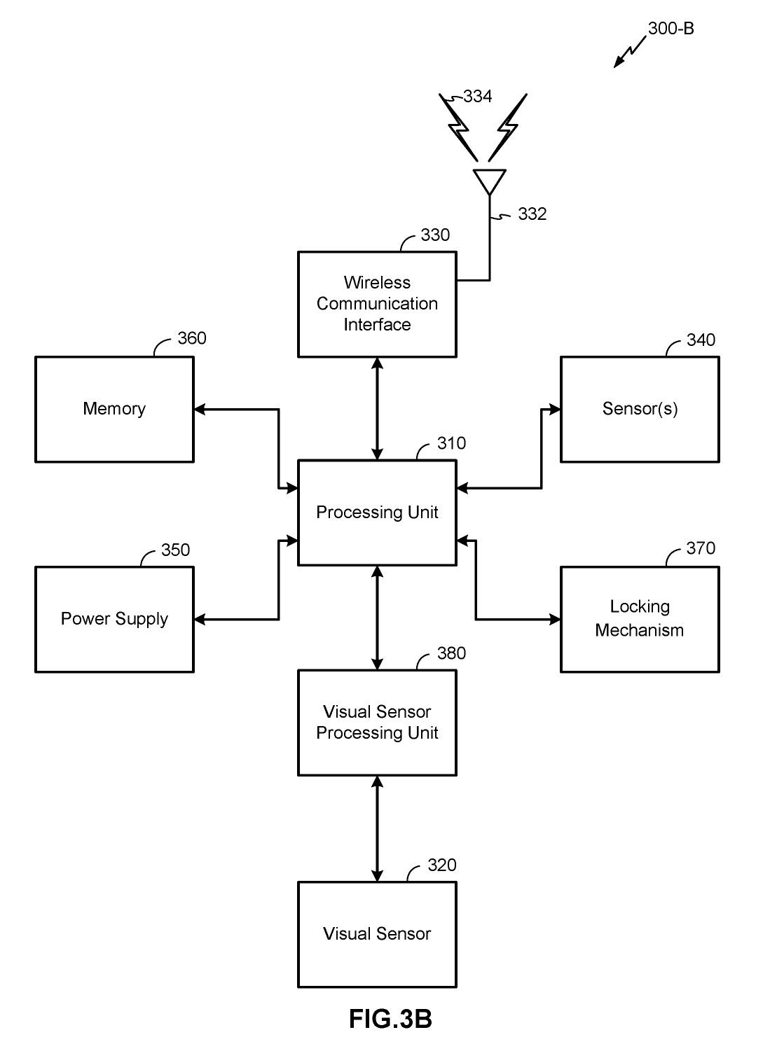

FIG. 3A is a simplified block diagram 300-A of the electrical components of an inhaler 110. As previously indicated, these components can be housed in or coupled to the inhaler 110 and/or otherwise incorporated therein. These components comprise a processing unit 310, a visual sensor 320, a power supply 350, a memory 360, sensor(s) 340, a wireless communication interface 330, and an antenna 332. Arrows between components illustrate communication and/or power links, which may be implemented by one or more buses and/or power lines. As with other figures provided herein, the embodiment illustrated here is an example. Other embodiments are not so limited.

The processing unit 310 can be used to process information provided by the visual sensor 320 and/or sensor(s) 340, as well as orchestrate the functionality of the various components illustrated in FIG. 3A. The processing unit 310 may comprise without limitation one or more general-purpose processors, one or more special-purpose processors (such as digital signal processing (DSP) chips, graphics acceleration processors, application specific integrated circuits (ASICs), and/or the like), and/or other processing structure or means, which can be configured to perform one or more of the methods described herein. Because an inhaler can have a long shelf life, the processing unit may be configured to operate in an extremely low power mode that, along with the capacity of the power supply 350, can allow the electrical components of the inhaler to be used in after a substantially long time (e.g., two years or longer) of no use.

Sensor(s) 340 can include one or more sensors configured to provide measurements and/or other information that may complement information provided by the visual sensor 320. For example, the sensor(s) 340 may comprise a clock or other means to determine time and date medicine was administered. As such, data provided by the inhaler can include a time/date stamp associated with the information provided by the inhaler. (Alternatively, a clock may be incorporated into the processing unit 310.)

The sensor(s) 340 may additionally or alternatively comprise a sensor to determine that medicine was administered. This can include a button pressed by the user when administering the drug or a sensor incorporated into and/or otherwise configured to determine the activation of a physical mechanism associated with the administration of the drug.

The sensor(s) 340 may additionally or alternatively include one or more inertial sensors such as accelerometers and/or gyroscopes to determine the orientation of the inhaler when used. Because the proper use of an inhaler often requires the inhaler to be oriented vertically (as illustrated in FIG. 2), the one or more accelerometers and/or gyroscopes can be used to confirm whether the inhaler had proper orientation when the drug was administered. Confirmation of proper (e.g., vertical) orientation may include obtaining an angle of the inhaler using the one or more accelerometers and/or gyroscopes, and determining that the angle is within a threshold degree of angle variation from a true vertical orientation.

The sensor(s) 340 may additionally or alternatively include one or more microphones. Microphones can be used to detect if the user is inhaling during the administration of the drug and (optionally) the duration of an inhalation. This timing and duration of inhalation can be another indicator of proper use of the inhaler. For example, if data from the microphone suggests an inhalation took place within a threshold amount of time (before and/or after) from the time the drug was administered, and/or the inhalation took place for at least a threshold amount of time, this may indicate proper inhalation. Proper inhalation can be used with data from other sensors to determine whether the inhaler was properly used.

The sensor(s) 340 may additionally or alternatively include one or more ambient light sensors. Ambient light sensors may be located near the orifice 230 of an inhaler, and may be covered by the user's mouth during proper use of the inhaler. Accordingly, the ambient light sensors may provide information indicative of proper use if, during the administration of the drug, the ambient light sensors sensed no or little light (e.g., when the orifice is in the mouth of the user), but after and/or before the administration of the drug (e.g., when the orifice 230 is not in the mouth of the user) a larger amount of light is detected. In some embodiments, a second visual sensor 320 may be used as an ambient light sensor.

The sensor(s) 340 may additionally or alternatively include one or more lip sensors. Similar to the ambient light sensors, lip sensors may be located on or near the orifice and can determine that the user's lips or mouth are on the orifice. Such sensors can employ circuitry and/or other means to measure the presence of a lip by, for example, measuring a skin's resistance, conductance, and/or capacitance (e.g., when the skin of the mouth and/or lips is in contact with the one or more lip sensors).

The memory 360 may comprise, without limitation, local accessible storage (e.g., a solid-state storage component) and may include instructions executed by the processing unit 310. In some embodiments the processing unit 310 may fully incorporate and/or otherwise include memory 360. In some embodiments, the memory 360 may be programmable, flash-updateable, and/or the like. Such memory 360 may be configured to implement any appropriate data stores, including without limitation, various file systems, database structures, and/or the like. This memory 360 may be used to store the messages and/or other information generated at or sent to/from the inhaler 110 and the connecting device 130, as described herein, which can be implemented using a database, linked list, or any other type of data structure. Additionally or alternatively, the messages and/or other information may be stored in a separate memory utilized by dedicated hardware for data collection.

The memory 360 of the apparatus 300 also can comprise software elements (not shown), including an operating system, device drivers, executable libraries, and/or other code, such as one or more application programs, which may comprise computer programs provided by various embodiments, and/or may be designed to implement methods, and/or configure systems, provided by other embodiments, as described herein. Merely by way of example, one or more procedures described with respect to the functionality discussed above might be implemented as machine-readable code and/or instructions executable by the processing unit 310.

The power supply 350 can comprise a battery and/or other source of power for the processing unit 310 and/or other components illustrated in FIG. 3A. In some embodiments, the power supply 350 can comprise a lithium-based battery or other battery type configured to have a relatively long shelf life, thereby enabling the electrical components of the inhaler to function after long periods without use. The capacity of the power supply 350 can be determined by the power needs of the electrical components, the expected shelf life of the inhaler, the expected number of uses of the inhaler, and/or other factors.

The wireless communication interface 330 may comprise a wireless transceiver and/or a chipset (such as a Bluetooth®, Bluetooth® low energy (BLE) device, an IEEE 802.15.x (e.g., Zigbee®) device, etc.), and/or the like. The wireless communication interface 330 may permit data to be communicated with a communication device (such as the connecting device 130 of FIG. 1) and/or any other electronic devices described herein. The communication can be carried out via one or more wireless communication antenna(s) 332 that send and/or receive wireless signals 334.

The locking mechanism 370 can allow the inhaler to prevent the improper administration of the drug, based on inputs from the visual sensor 320 and/or sensor(s) 340. The locking mechanism 370 may be controllable by the processing unit 310, and may prevent a physical and/or electrical actuator from administrating the drug of the inhaler. For example, the processing unit 310 may use the locking mechanism 372 prohibit administration of the drug when a face is not detected and/or recognized by the visual sensor 320, an improper orientation of the inhaler is detected, and/or other factors that would indicate the improper use of the inhaler (if the drug is administered) are present.

The visual sensor 320 can comprise a camera or other device capable of facial detection and/or recognition. For example, a visual sensor 320 may be used that integrates a special-purpose camera with dedicated computer vision (CV) computation hardware and a dedicated low-power microprocessor for the purposes of detecting, tracking, recognizing, and/or analyzing subjects, objects, and scenes in the view of the camera, including the face of a user of the inhaler. As indicated elsewhere herein, some embodiments may include a plurality of visual sensors, which may be positioned at different locations on the inhaler to capture different visual information. The visual sensor 320 may process the information retrieved from the camera using the included low-power microprocessor and send indications that one or more reference occurrences (such as the detection or recognition of face) have occurred to the processing unit 310. This can allow the processing unit 310 to stay in a low-power (e.g. “sleep”) state most of the time. Alternatively, as illustrated in FIG. 3B, a visual sensor 320 may not incorporate a low-power processing unit, but may have a separate visual sensor processing unit 380 configured to provide preliminary processing of data captured by the visual sensor 320. In such embodiments, the visual sensor processing unit 380 may provide optimality similar to a low-power microprocessor integrated into a visual sensor 320, as described above. For example, the visual sensor processing unit 380 may process information obtained by the visual sensor 320 and send indications that one or more reference occurrences have occurred to the processing unit 310, enabling the processing unit 310 to stay in a low-power state until it receives the indications that the one or more reference occurrences have occurred. In some embodiments having multiple visual sensors 320, a single visual sensor processing unit 380 may be communicatively coupled with the multiple visual sensors 320. Referring again to FIG. 3A, some embodiments may comprise a visual sensor 320 without an internal low-power processing unit, but instead the processing unit 310 performs the preliminary processing of data while using sufficiently low power to enable the electrical components of the inhaler to operate after substantially long periods without use. In some embodiments, the visual sensor 320 may simply comprise a camera, where visual processing of image data captured by the camera may be performed by a processing unit of the inhaler (e.g., processing unit 310 and/or visual sensor processing unit 380) and/or on a remote device (e.g., the connecting device, a hub, a server, etc.). In some embodiments, the inhaler (or other mobile medical dispenser) can use data received (e.g., via the wireless communication interface 330) from one or more remote cameras (or other visual sensors) in addition to the visual sensor 320.

An example output of the visual sensor 320 can include any of a variety of indications, depending on desired functionality. In some embodiments, for example, the visual sensor 320 can simply provide an output indicating that a face is currently detected. More sophisticated outputs may be provided, such as a location of the face, an orientation of the face and/or one or more facial features, a position or size of the bounding box of the face (and/or one or more facial features), and identity of a person recognized using facial recognition, a number of faces detected, a distance to a mouth, the opening of a mouth, and/or the like. It can be noted that, in the embodiments described herein, the detection of a “face” may comprise detection of one or more facial features. Thus, facial detection as used herein may not necessarily comprise detection of an entire face, but may instead include a portion of a face (including one or more facial features).

An example of a visual sensor 320 that could be utilized in the configurations of FIGS. 3A and 3B is described in further detail in U.S. patent application Ser. No. 14/866,549, entitled “Low-Power Always-On Face Detection, Tracking, Recognition and/or Analysis Using Evidence-Based Vision Sensor,” filed Sep. 25, 2015, which is assigned to the assignee hereof and Incorporated by reference herein for all purposes.

Depending on the power consumption of the visual sensor 320, the operation of the visual sensor 320 may vary. In some embodiments, for example, the visual sensor 320 may be always on (e.g., capture images periodically at a given rate, without interruption). In other embodiments, triggering activities (the press of a button, sensor data indicating the opening of a package of the inhaler, etc.) may cause the visual sensor 320 to power on and begin capturing visual information.

The visual information captured by the visual sensor 320 can vary, depending on desired functionality. As indicated previously, the visual sensor 320 may simply determine whether or not one or more facial features are present within its field of view (e.g., the field of view of its camera). When this determination is made, the visual sensor 320 can provide an indication of the determination to the processing unit 310, which can use information from the sensor(s) 340 at substantially the same time to determine other factors, such as the orientation of the inhaler, administration of medicine, insertion of the orifice of the inhaler into a mouth, and the like. The processing unit can then determine a likelihood that the inhaler was used properly (or, more generally, an effectiveness of how the drug was administered), and provide an indication of that likelihood to a communication device and/or remote device (which, in turn, provides the information to the stakeholder(s)) via the wireless communication interface 330. Additionally or alternatively, the processing unit 310 can provide information from the visual sensor 320 and sensor(s) 340 to the communication device and/or remote device, which makes the determination of the likelihood that the inhaler was used properly.

The visual sensor 320 can provide additional or alternative information, according to some embodiments. In some embodiments, for example, the visual sensor 320 can provide facial recognition. This may require some information provided via the wireless communication interface 330 (e.g., from the communication device) to allow for recognition of a particular user (such as facial feature information, and the like). The communication device can, for example, execute an application in which it obtains features for facial recognition using a camera, and provides information indicative of these features to the inhaler via the wireless communication interface 330. Such functionality can enable the visual sensor to further provide an indication regarding whether the face of the proper user was within its field of view during the administration of the drug.

The visual sensor 320 may additionally or alternatively detect other elements, which may be utilized by the processing unit 310 to determine proper use of the inhaler. The visual sensor can, for example, determine where, within its field of view, one or more facial features are detected (which can help ensure the user has properly inserted the orifice of the inhaler into the user's mouth), an emotion or facial expression of the user (which can help determine whether the user is inhaling the medicine), a proximity of a detected face, a length of time during which a face was detected and/or within a certain proximity, an orientation of the face relative to the inhaler, an ambient environment of the inhaler, the presence of objects other than a face, and the like. Any or all of which may be used to inform the determination of whether administration of the drug by the inhaler was proper.

As noted above, information provided by the visual sensor 320 and the sensor(s) 340 can be processed by the processing unit 310 (or sent to a processing unit of the communication device 320) to make a determination regarding the likelihood that, when administration of the drug is detected, the inhaler administered the drug properly. This determination can be based on the “fusion” of sensor data to provide a larger picture of whether the drug was administered properly. For example, the visual sensor 320 may indicate that a face was detected during the administration of the drug, but an accelerometer may indicate that the inhaler was not oriented vertically (or within a threshold angle from a vertical position), and a visual sensor 320 indicates that the face is only detected for three seconds, where proper administration of the drug requires inhalation for five seconds. Given these factors, which may be weighted differently, the processing unit 310 can make a determination of the likelihood that the drug was administered properly. An indication of the determination may be provided via a binary output (administered properly or not), made on a scale (e.g., a scale of 1 to 10 where 10 reflects the highest likelihood that the drug was administered properly), and/or in some other manner. Where stakeholder receives a determination that the drug was not administrated properly, the stakeholder may contact the user for additional information to determine whether it was administered properly, and why or why not.

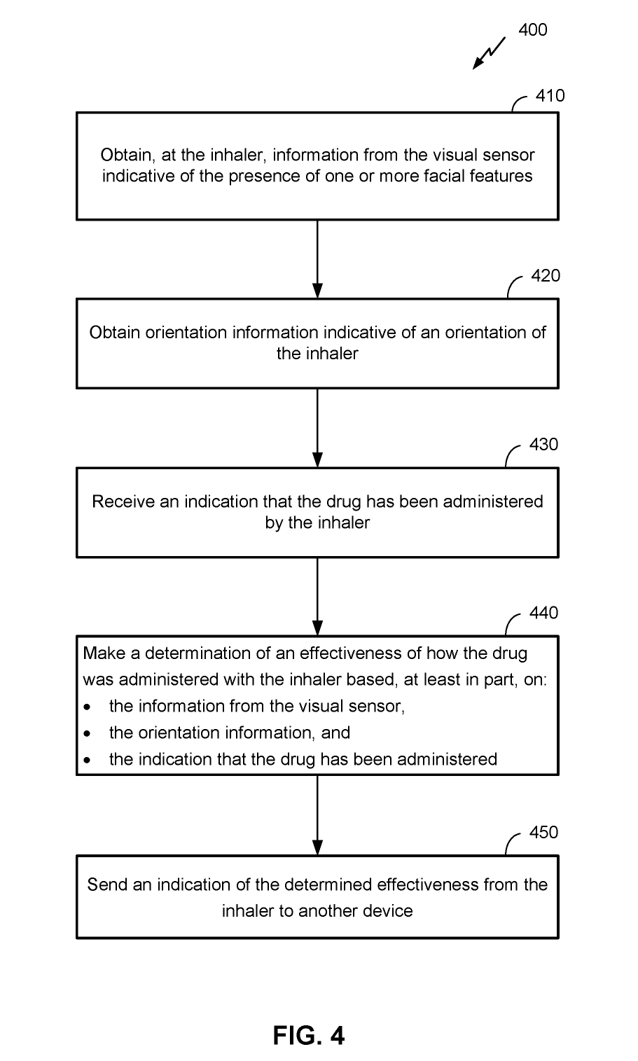

FIG. 4 is a flow diagram 400 illustrating an embodiment of a method of operating an inhaler with a visual sensor, according to one embodiment. It can be noted, however, that the embodiment provided in FIG. 4 is a non-limiting example. A person of ordinary skill in the art will recognize alternative embodiments may employ any of a variety of variations. Any or all of the functions illustrated in the boxes of FIG. 4 can be performed by an inhaler (such as the inhalers 110 of FIGS. 1-3B and/or other inhalers described in embodiments provided herein), although alternative embodiments may enable additional or alternative devices to perform some or all of the functions.

At block 410, information indicative of the presence of one or more facial features is obtained at the inhaler from the visual sensor. As described earlier, a visual sensor may be disposed on an inhaler such that it is able to capture an image of a face (of the user of the inhaler) during proper use of the inhaler (as well as before and after, according to some embodiments). This information may be obtained before, during, and/or after it is determined (e.g., using sensor data) that the inhaler is used to administer medicine. In some embodiments, the visual sensor may further provide information regarding the relative orientation, size, proximity, or location of the one or more facial features, the time duration of facial proximity, and/or other detected features. In some embodiments, the placement (and the duration of placement) into the mouth of a user may be detected by ambient light sensing (e.g., by a second visual sensor placed on the inhaler at a location that is placed within the user's mouth during proper usage of the inhaler). In some embodiments, the visual sensor may not only detect that a face is present, but recognize a user's identity (e.g., perform facial recognition based on facial features). In such cases, the information indicative of the presence of one or more facial features may therefore comprise information indicative of a facial recognition event. As indicated previously, any of a variety of other sensors may be utilized to determine whether the inhaler is used to administer medicine. Means for performing the functionality at block 410 can include, for example, the processing unit 310, visual sensor 320, power supply 350, memory 360, and/or other components of an inhaler 300, as illustrated in FIGS. 3A-3B and described above.

At block 420, orientation information indicative of an orientation of the inhaler is obtained. As noted above, this may be obtained by the visual sensor and/or other sensors, such as one or more accelerometers and/or gyroscopes. According to embodiments, the orientation information may include information regarding whether absolute orientation was proper (e.g., whether the inhaler is vertical—or within a threshold angle of a vertical position—with respect to the earth) and/or whether orientation was proper with respect to a user. Depending on embodiments, this information may be obtained based on a triggering event, such as an indication that the drug has been administered by the inhaler, the presence of one or more facial features have been detected by the visual sensor, and/or other triggers that past, present, or imminent future use of the inhaler. Means for performing the functionality at block 420 can include, for example, the processing unit 310, visual sensor 320, sensor(s) 340, power supply 350, memory 360, and/or other components of an inhaler 300, as illustrated in FIGS. 3A-3B and described above.

At block 430, an indication that the drug has been administered by the inhaler is received. This information, too, may be obtained via one or more sensors. For instance, a sensor may be coupled with the mechanism for administering the drug, detecting physical movement, sound, pressure, fluid flow, and/or other characteristic(s) indicating drug administration. This information may be obtained based on a triggering event indicative of imminent administration of the drug. In some embodiments, the method may include releasing a locking mechanism of the inhaler to enable administration of the drug prior to the functionality at block 430, in response to obtaining the information from the visual sensor regarding the presence of the one or more facial features. Means for performing the functionality at block 430 can include, for example, the processing unit 310, visual sensor 320, sensor(s) 340, power supply 350, memory 360, and/or other components of an inhaler 300, as illustrated in FIGS. 3A-3B and described above.

At block 440, a determination is made of an effectiveness of how the drug was administered with the inhaler based, at least in part, on the information from the visual sensor, the orientation information, and the indication that the drug has been administered. As noted above, the determination of the effectiveness of how the drug was administered with the inhaler may be performed by a processor that awakens from a low-power mode based, at least in part, on the obtaining the information from the visual sensor indicative of the presence of the one or more facial features. In some embodiments, the determination of the effectiveness may be triggered, for example, by receiving the indication that the drug has been dispensed by the inhaler, and the effectiveness itself may be determined from the orientation information and information from the visual sensor, which may be weighted differently, depending on desired functionality.

In some embodiments, the effectiveness may further be determined from additional information that may be obtained from the visual sensor and/or other sensors, as indicated previously herein. Such additional information may include, for example, distance to mouth, mouth opening, time of day, user orientation relative to the device, etc. Some information may be collected before and/or after the administration of the medicine. For example, an inhaler may track facial features after the medicine is administered to determine whether the user may be closing his or her mouth too soon after the medication has been dispensed. Additionally or alternatively, the determination of the effectiveness of how the drug was administered with the inhaler may be further based on information from the microphone, which may be indicative of timing and/or duration of inhalation. In some embodiments, the determination of the effectiveness may also be based on from information received from a light sensor and/or a sensor configured to measure resistance, conductance, and/or capacitance.

Means for performing the functionality at block 440 can include, for example, the processing unit 310, visual sensor 320, power supply 350, sensor(s) 340, memory 360, and/or other components of an inhaler 300 as illustrated in FIGS. 3A-3B and described above.

At block 450, an indication of the determined effectiveness is sent from the inhaler to another device. As noted above in relation to FIG. 1, this indication may be sent wirelessly to a connecting device and/or one or more stakeholders via a communication network. The indication of the determination of whether the drug was properly administered (e.g., the effectiveness of the administration of the drug) may be binary, on a numerical scale, or indicated by some other means, and may be sent to a connecting device and ultimately to any of a variety of stakeholders. In some embodiments, the inhaler may further send an indication of a time at which the drug was administered (e.g., a timestamp). As indicated herein, the determination of whether a drug was properly administered with the inhaler may require a synchronization of output signals of the visual sensor with those of other sensors of the inhaler. Means for performing the functionality at block 450 can include, for example, the processing unit 310, wireless communication interface 330, power supply 350, sensor(s) 340, memory 360, and/or other components of an inhaler 300 as illustrated in FIGS. 3A-3B and described above.

It can be noted that, although embodiments provided herein are limited to an inhaler, other embodiments are not so limited. Applications of the utilization of a visual sensor in the manner described herein can be found in other industries and contexts. For example, the techniques provided herein can be utilized in any device for administration of a drug by inhalation or other means at a certain location of the body (which can have a naturally distinctive feature (such as a face) or an artificial feature (such as a tattoo, skin patch, etc.)). The location of the body can be detected by a visual sensor, and the detection may be used to make a determination of whether the administration of the drug was at the correct body location, and/or successful or proper.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus, many of the elements are examples that do not limit the scope of the disclosure to those specific examples.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein.

Therefore, it is intended that claimed subject matter not be limited to the particular examples disclosed, but that such claimed subject matter may also include all aspects falling within the scope of appended claims, and equivalents thereof.

What is claimed is:

1. A method of operating an inhaler with a visual sensor, the method comprising:
obtaining, at the inhaler, information from the visual sensor indicative of the presence of one or more facial features;
obtaining orientation information indicative of an orientation of the inhaler;
receiving an indication that a drug has been administered by the inhaler;
making a determination of an effectiveness of how the drug was administered with the inhaler based, at least in part, on:
the information from the visual sensor,
the orientation information, and
the indication that the drug has been administered; and
sending an indication of the determined effectiveness from the inhaler to another device.

2. The method of claim 1, wherein:
the information regarding the presence of one or more facial features further comprises information regarding orientation, size, proximity, or location of the one or more facial features, or any combination thereof.

3. The method of claim 1, further comprising sending, from the inhaler to the other device, an indication of a time at which the drug was administered.

4. The method of claim 1, wherein the orientation information is obtained from an accelerometer, a gyroscope, and/or the visual sensor.

5. The method of claim 1, wherein making the determination of the effectiveness of how the drug was administered with the inhaler is further based on information from a microphone.

6. The method of claim 5, wherein the information from the microphone is indicative of timing of inhalation, duration of inhalation, or both.

7. The method of claim 1, wherein making the determination of the effectiveness of how the drug was administered with the inhaler is further based on information received from a light sensor.

8. The method of claim 1, wherein making the determination of the effectiveness of how the drug was administered with the inhaler is further based on information received from a sensor configured to measure resistance, conductance, capacitance, or any combination thereof.

9. The method of claim 1, further comprising, in response to obtaining the information from a visual sensor regarding the presence of one or more facial features, releasing a locking mechanism of the inhaler to enable administration of the drug.

10. The method of claim 9, wherein the information indicative of the presence of the one or more facial features comprises information indicative of a facial recognition event.

11. The method of claim 1, wherein making the determination of the effectiveness of how the drug was administered with the inhaler is performed by a processor that awakens from a low-power mode based, at least in part, on the obtaining the information from the visual sensor indicative of the presence of the one or more facial features.

12. The method of claim 1, wherein the indication of the determined effectiveness is based on a numerical scale.

13. An inhaler comprising:
a visual sensor configured to visually detect the presence of one or more facial features of a person using the inhaler;
an orientation sensor configured to determine an orientation of the inhaler;
a wireless communication interface; and
a processing unit communicatively coupled with the visual sensor, the orientation sensor, and the wireless communication interface and configured to:
obtain, from the visual sensor, an indication of the presence of the one or more facial features;
obtain, from the orientation sensor, orientation information indicative of the orientation of the inhaler;
receive an indication that a drug has been administered by the inhaler;
make a determination of an effectiveness of how the drug was administered with the inhaler based, at least in part, on:
the information from the visual sensor,
the orientation information, and
the indication that the drug has been administered; and
send an indication of the determined effectiveness, via the wireless communication interface, from the inhaler to another device.

14. The inhaler of claim 13, wherein the visual sensor is configured to provide information regarding the presence of one or more facial features that comprises information regarding orientation, size, proximity, or location of the one or more facial features, or any combination thereof.

15. The inhaler of claim 13, wherein the processing unit is further configured to send, from the inhaler to the other device, an indication of a time at which the drug was administered.

16. The inhaler of claim 13, wherein the orientation sensor comprises an accelerometer, a gyroscope, and/or the visual sensor.

17. The inhaler of claim 13, further comprising a microphone, wherein the processing unit is further configured to make the determination of the effectiveness of how the drug was administered with the inhaler further based on information from the microphone.

18. The inhaler of claim 17, wherein the information from the microphone is indicative of timing of inhalation, duration of inhalation, or both.

19. The inhaler of claim 13, further comprising a light sensor, wherein the processing unit is further configured to make the determination of the effectiveness of how the drug was administered with the inhaler further based on information received from the light sensor.

20. The inhaler of claim 13, further comprising a skin-detection sensor configured to measure resistance, conductance, capacitance, or any combination thereof, wherein the processing unit is further configured to make the determination of the effectiveness of how the drug was administered with the inhaler further based on information received from a sensor.

21. The inhaler of claim 13, further comprising a locking mechanism, where in the processing unit is further configured to, in response to obtaining the information from a visual sensor regarding the presence of one or more facial features, release the locking mechanism to enable administration of the drug.

22. The inhaler of claim 13, wherein the processing unit is configured to awaken from a low-power mode based, at least in part, on the obtaining the information from the visual sensor indicative of the presence of the one or more facial features.

23. The inhaler of claim 13, wherein the processing unit is configured to determine the indication of the determined effectiveness based on a numerical scale.

24. A drug-administering apparatus comprising:
means for obtaining information from visual-sensing means indicative of the presence of one or more facial features;
means for obtaining orientation information indicative of an orientation of the drug-administering apparatus;
means for receiving an indication that a drug has been administered by the drug-administering apparatus;
means for making a determination of an effectiveness of how the drug was administered with the drug-administering apparatus based, at least in part, on:
the information from the visual-sensing means,
the orientation information, and
the indication that the drug has been administered; and
means for wirelessly sending an indication of the determined effectiveness from the drug-administering apparatus to a separate device.

25. The drug-administering apparatus of claim 24, further comprising means for sending, from the drug-administering apparatus to the separate device, an indication of a time at which the drug was administered.

26. The drug-administering apparatus of claim 24, wherein the means for making the determination of the effectiveness of how the drug was administered with the administering apparatus comprise means for further basing the determination on information received from means for detecting sound.

27. The drug-administering apparatus of claim 24, wherein the means for making the determination of the effectiveness of how the drug was administered with the administering apparatus further comprise means for further basing the determination on information received from means for detecting light.

28. The drug-administering apparatus of claim 24, wherein the means for making the determination of the effectiveness of how the drug was administered with the administering apparatus further comprise means for further basing the determination on information received from means for detecting skin.

29. The drug-administering apparatus of claim 24, further comprising, means for, in response to obtaining the information from a visual sensor regarding the presence of one or more facial features, releasing a locking mechanism of the drug-administering apparatus to enable administration of the drug.

30. A non-transitory computer-readable medium having instructions embedded thereon for operating an inhaler with a visual sensor, the instructions comprising computer code for:
obtaining information from the visual sensor indicative of the presence of one or more facial features;
obtaining orientation information indicative of an orientation of the inhaler;
receiving an indication that a drug has been administered by the inhaler;
making a determination of an effectiveness of how the drug was administered with the inhaler based, at least in part, on:
the information from the visual sensor,
the orientation information, and
the indication that the drug has been administered; and
sending an indication of the determined effectiveness from the inhaler to another device.
